(19) United States
(12) Patent Application Publication
(10) Pub. No.: US 2014/0121234 A1
(43) Pub. Date: May 1, 2014

(54) INHALANT PROPELLANT-FREE AEROSOL FORMULATION

(71) Applicants: Christoph KREHER, Ingelheim am Rhein (DE); Michael SPALLEK, Ingelheim am Rhein (DE)

(72) Inventors: Christoph KREHER, Ingelheim am Rhein (DE); Michael SPALLEK, Ingelheim am Rhein (DE)

(73) Assignee: BOEHRINGER INGELHEIM INTERNATIONAL GMBH, Ingelheim am Rhein (DE)

(21) Appl. No.: 14/146,624

(22) Filed: Jan. 2, 2014

Related U.S. Application Data
(63) Continuation of application No. 12/301,292, filed on Feb. 13, 2009, now abandoned.

(30) Foreign Application Priority Data
May 20, 2006 (DE) 10 2006 023 770.6

(51) Int. Cl. A61K 9/00; A61K 31/439; A61K 31/137

(52) U.S. Cl. CPC A61K 9/0078; A61K 31/137; A61K 31/439
USPC 514/291; 514/653; 514/304

(57) ABSTRACT

The invention relates to inhalant propellant-free aerosol formulations containing at least one inert, non-volatile auxiliary substance for adjusting defined droplet sizes.

The present invention relates to propellant-free aerosol formulations for inhalation containing one or more inert, non-volatile excipients for adjusting defined droplet sizes.

### BACKGROUND TO THE INVENTION

Medicaments are administered by inhalation not exclusively but especially when treating respiratory ailments such as asthma or COPD, for example. Suitable medicament formulations, besides powdered formulations and propellant-containing aerosol formulations, also include in particular propellant-free aqueous or aqueous-alcoholic solutions of active substances.

Propellant-free solution formulations of this kind are known in the art. Ethanolic formulations are disclosed for example by WO 97/01329, while aqueous systems are described for example by WO 98/27959.

In the administration of medicaments by inhalation, irrespective of the choice of formulation, the size of the particles to be inhaled is of particular importance. Only particles within a certain range of sizes are able to reach the intended site of activity, the deep branches of the lungs, to develop the desired therapeutic effect therein. On the one hand, the particles to be inhaled should not exceed a certain upper limit. Moreover, in order to achieve optimum deposition in the lungs, it is desirable that the particle size of an aerosol does not fall below a certain lower limit. Preferably, the particle size of the aerosol should be more than 0.5 μm, for alveolar deposition preferably more than 1 μm and for bronchial deposition particularly preferably more than 2 μm.

Particularly volatile aerosols have the property that their droplets evaporate during inhalation and their diameter decreases sharply as a result.

The aim of the present inventions is to provide formulations aerosol solutions in which the minimum particle size of the aerosol ingredients delivered has a lower limit.

### DETAILED DESCRIPTION OF THE INVENTION

Surprisingly it has been found that this aim can be achieved if inert non-volatile excipients are added to the aerosol solution formulations.

Accordingly, the present invention relates to propellant-free solution formulations for inhalation which contain in addition to one or more active substances in a solvent selected from among water, ethanol and water-ethanol mixtures, at least one inert, non-volatile excipient in an amount such that the total concentration of non-volatile constituents in the formulation is ≧3 wt. %.

For the purposes of the present invention all the suspended or dissolved constituents that would be left after evaporation of the solvent contribute to the total concentration of non-volatile constituents. These are, primarily, inert non-volatile excipients, besides the active substance ingredients and any preservatives and complexing agents present.

Within the scope of the formulations according to the invention, inert non-volatile excipients are compounds which may be present in dissolved, partly dissolved or suspended form in the solvent of the formulations according to the invention. Examples of inert non-volatile excipients which may be used within the scope of the solution formulations according to the invention include for example monosaccharides, disaccharides, oligo- and polysaccharides, polyalcohols or salts.

Monosaccharides are preferably selected from among glucose and arabinose, of which glucose is particularly preferred. Disaccharides are preferably selected from among lactose, saccharose, maltose and trehalose, while of the disaccharides lactose is particularly preferred. Oligo- and polysaccharides are preferably selected from among the dextranes. Polyalcohols are preferably selected from among sorbitolol, mannitol, xylitol and glycerol while of the polyalcohols glycerol is particularly preferred. Salts are preferably selected from among potassium chloride, magnesium chloride, magnesium sulphate, sodium chloride, sodium citrate, sodium phosphate, sodium hydrogen phosphate, sodium hydrogen carbonate, potassium citrate, potassium phosphate, potassium hydrogen phosphate, potassium hydrogen carbonate, calcium carbonate and calcium chloride, while, of the salts, sodium chloride, magnesium sulphate, sodium hydrogen carbonate, potassium hydrogen carbonate and magnesium chloride, especially sodium chloride, magnesium sulphate and sodium hydrogen carbonate are particularly preferred.

The formulations according to the invention may also contain ethereal oils as mucoactive substances for flavour masking. Of particular interest in this respect are eucalyptus oil, silver fir oil, pine-needle oil or peppermint oil.

The inert, non-volatile excipients used are particularly preferably compounds selected from among the above-mentioned salts, while the following excipients are of particular significance: sodium chloride. Sodium chloride, magnesium sulphate, sodium hydrogen carbonate, potassium hydrogen carbonate and magnesium chloride.

Preferably the total concentration of non-volatile constituents of the solution formulations according to the invention is ≧3 wt. %, particularly preferably ≧5 wt. %, more preferably ≧10 wt. %. The symbol “≧” represents “equal to or more than” such as for example “3 wt. % or more”.

The maximum concentration of non-volatile constituents should not exceed a range of 20 wt. %.

Particularly preferred are medicament preparations which, after being nebulised by a suitable inhaler, have particle sizes of ≧0.5 μm, for alveolar deposition preferably ≧1 μm and for bronchial deposition particularly preferably ≧2 μm.

The medicament formulations according to the invention contain as solvent pure water, pure ethanol or mixtures of ethanol and water. If ethanol-water mixtures are used, the percentage amount of ethanol by mass in these mixtures is preferably in the range between 5 and 99% ethanol, particularly preferably in the range from 10 to 96% ethanol. Most particularly preferred medicament formulations for the purposes of the present invention contain as solvent pure water, pure ethanol or ethanol-water mixtures containing between 50 and 92%, particularly preferably between 69 and 91% ethanol.

If desired, other co-solvents may be used besides ethanol and water. They are preferably selected from among the alcohols or ethers, such as for example isopropanol or tetrahydrofuran. According to the invention, however, it is preferable not to use an additional solvent.

Usually, the formulations according to the invention contain pharmacologically acceptable acids for adjusting the pH. The pH of the formulation according to the invention is preferably in the range from 2.0 to 6.5, preferably between 2.2 and 5.0, particularly preferably between about 2.5 and 4.5, according to the invention.

The pharmacologically acceptable acids used may be inorganic or organic acids. Examples of preferred inorganic acids are selected from among hydrochloric acid, hydrobromic acid, nitric acid, sulphuric acid and phosphoric acid. Examples of particularly suitable organic acids are selected from among ascorbic acid, citric acid, malic acid, tartaric acid, maleic acid, succinic acid, fumaric acid, acetic acid, formic acid and propionic acid. Preferred inorganic acids are hydrochloric acid and sulphuric acid, while hydrochloric acid is of particular importance according to the invention. Of the organic acids, ascorbic acid, fumaric acid and citric acid are preferred, while citric acid is particularly preferred according to the invention. Mixtures of the acids specified may optionally also be used, particularly in the case of acids which have other properties in addition to their acidifying properties, e.g. as flavourings or antioxidants, such as for example citric acid or ascorbic acid.

Pharmacologically acceptable bases may optionally also be used for precise titration of the pH. Suitable bases include for example alkali metal hydroxides and alkali metal carbonates. The preferred alkali metal ion is sodium. If such bases are used, care must be taken to ensure that the resulting salts which are then contained in the finished medicament formulation are pharmacologically compatible with the above-mentioned acid.

The formulations according to the invention may contain complexing agents or preservatives as other ingredients. By complexing agents are meant within the scope of the present invention molecules which are capable of entering into complex bonds. Preferably, these compounds should have the effect of complexing cations, most preferably metal cations. The formulations according to the invention preferably contain editic acid (EDTA) or one of the known salts thereof, e.g. sodium EDTA or disodium EDTA, as complexing agent. Preferably, disodium edetate is used, optionally in the form of its hydrates, more preferably in the form of its dihydrate. If complexing agents are used within the formulations according to the invention, their content is preferably in the range from 1 to 50 mg per 100 ml, more preferably in the range from 2 to 15 mg per 100 ml of the formulation according to the invention. Preferably, the formulations according to the invention contain a complexing agent in an amount of about 4 to 12 mg per 100 ml, more preferably about 10 mg per 100 ml of the formulation according to the invention.

The remarks made concerning disodium edetate also apply analogously to other possible additives which are comparable to EDTA or the salts thereof, which have complexing properties and can be used instead of them, such as for example nitrilotriacetic acid and the salts thereof.

Preservatives can be added to protect the formulation from contamination with pathogenic bacteria. Suitable preservatives are those known from the prior art, particularly benzalkonium chloride or benzoic acid or benzoates such as sodium benzoate in the concentrations known from the prior art. Preferably, benzalkonium chloride is added to the formulation according to the invention. The amount of benzalkonium chloride is between 1 mg and 50 mg per 100 ml of formulation, preferably about 2 to 15 mg per 100 ml, more preferably about 3 to 12 mg per 100 ml, particularly preferably about 4 to 10 mg per ml of the formulation according to the invention.

Benzalkonium chloride may also be used according to the invention in admixture with other preservatives.

The active substances that may be used within the scope of the formulations according to the invention are preferably selected from among the anticholinergics, betamimetics, corticosteroids, PDE4-inhibitors, LTD4-antagonists, dopamine agonists and H1-antihistamines; one or more of these active substances may be present in each case.

Anticholinergics which may be used as active substance in the medicament combinations according to the invention are preferably selected from among tiotropium salts, oxitropium salts, flutropium salts, ipratropium salts, glycopyrronium salts and trospium salts. In the above-mentioned bis salts the cations tiotropium, oxitropium, flutropium, ipratropium, glycopyrronium and trospium constitute the pharmacologically active ingredients. Any reference to the above-mentioned salts naturally includes a reference to the corresponding cations tiotropium, oxitropium, flutropium, ipratropium, glycopyrronium and trospium. By the bis salts are meant, according to the invention, those compounds which contain besides the cations tiotropium, oxitropium, flutropium, ipratropium, glycopyrronium and trospium, as the counter-ion (anion), the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts, the chlorides, bromides, iodides and methanesulphonates are particularly preferred.

Of particular importance are medicament combinations which contain tiotropium salts, oxitropium salts or ipratropium salts, while the respective bromides are of particular significance according to the invention. Of particular importance is tiotropium bromide. The above-mentioned salts may optionally be present in the medicament combinations according to the invention in the form of the solvates or hydrates thereof, preferably in the form of the hydrates. In the case of tiotropium bromide the medicament combinations according to the invention preferably contain it in the form of the crystalline tiotropium bromide monohydrate which is known from WO 02/30928.

The above-mentioned anticholinergics optionally have chiral carbon centres. In this case the medicament combinations according to the invention may contain the anticholinergics in the form of their enantiomers, mixtures of enantiomers or racemates, while enantiomerically pure anticholinergics are preferably used.

If tiotropium salts are used, the concentration of tiotropium cation in the medicament formulations according to the invention is preferably between 0.01 g per 100 g formulation and 0.06 g per 100 g formulation. An amount of 0.015 g/100 g to 0.055 g/100 g is preferred, while an amount of 0.02 g/100 g to 0.05 g/100 g is more preferred. Most preferred is an amount of 0.023±0.001 g per 100 g formulation to 0.045±0.001 g per 100 g formulation.

If ipratropium salts are used, the concentration of ipratropium cation in the medicament formulations according to the invention is preferably between 0.20 g per 100 g formulation and 1.58 g per 100 g formulation. An amount of 0.30 g/100 g to 1.45 g/100 g is preferred, an amount of 0.40 g/100 g to 1.32 g/100 g is more preferred. Most preferred is an amount of 0.46±0.02 g per 100 g formulation to 0.92±0.02 g per 100 g formulation.

If oxitropium salts are used, the concentration of oxitropium cation in the medicament formulations according to the invention is preferably between 0.20 g per 100 g formulation and 1.58 g per 100 g formulation. An amount of 0.30 g/100 g to 1.45 g/100 g is preferred, an amount of 0.40 g/100 g to 1.32 g/100 g is more preferred. Most preferred is an amount of 0.46±0.02g per 100 g formulation to 0.92±0.02 g per 100 g formulation.

In another preferred embodiment of the present invention the anticholinergics contained in the medicament combinations according to the invention are selected from the salts of formula

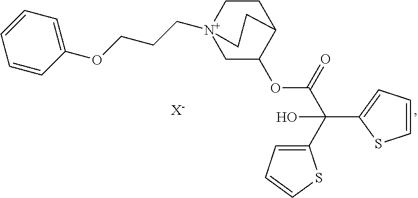

wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably bromide,
   optionally in the form of the racemates, enantiomers or hydrates thereof.

Of particular importance are those medicament combinations which contain the enantiomers of formula

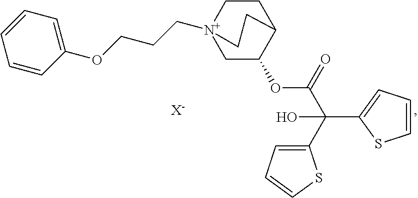 [SMILES: O=C(O[C@@H]1C[N+]2(CCCOc3ccccc3)CCC1CC2)C(O)(c1cccs1)c1cccs1 ; "X-"]

wherein X⁻ may have the meanings stated above.

The concentration in which the above-mentioned anticholinergics are present in the medicament preparations according to the invention is about 4 to 2000 mg per 100 g, preferably about 8 to 1600 mg per 100 g according to the invention. Particularly preferably, 100g of the formulations according to the invention contain about 80 to about 1360 mg of the above-mentioned anticholinergics (based on pharmacologically active cation). If the above-mentioned bromides are used, the concentration thereof in the compositions according to the invention is usually about 5 to 2500 mg per 100 g, preferably about 10 to 2000 mg per 100 g of medicament preparation. Particularly preferably, 100g of the formulations according to the invention contain about 100 to 1700 mg of one of the above-mentioned bromides.

In another preferred embodiment of the present invention are the anticholinergics contained in the preparations according to the invention are selected from among tropenol 2,2-diphenylpropionate methobromide, scopine 2,2-diphenylpropionate methobromide, scopine 2-fluoro-2,2-diphenylacetate methobromide, tropenol 2-fluoro-2,2-diphenylacetate methobromide, tropenol 3,3′,4,4′-tetrafluorobenzilate methobromide, scopine 3,3′,4,4′-tetrafluorobenzilate methobromide, tropenol 4,4′-difluorobenzilate methobromide, scopine 4,4′-difluorobenzilate methobromide, tropenol 3,3′-difluorobenzilate methobromide, scopine 3,3′-difluorobenzilate methobromide, tropenol 9-hydroxy-fluorene-9-carboxylate methobromide, tropenol 9-fluoro-fluorene-9-carboxylate methobromide, scopine 9-hydroxy-fluorene-9-carboxylate methobromide, scopine 9-fluoro-fluorene-9-carboxylate methobromide, tropenol 9-methyl-fluorene-9-carboxylate methobromide, scopine 9-methyl-fluorene-9-carboxylate methobromide, cyclopropyltropine benzilate methobromide, cyclopropyltropine 2,2-diphenylpropionate methobromide, cyclopropyltropine 9-hydroxy-xanthene-9-carboxylate methobromide, cyclopropyltropine 9-methyl-fluorene-9-carboxylate methobromide, cyclopropyltropine 9-methyl-xanthene-9-carboxylate methobromide, cyclopropyltropine 9-hydroxy-fluorene-9-carboxylate methobromide, cyclopropyltropine methyl 4,4′-difluorobenzilate methobromide, tropenol 9-hydroxy-xanthene-9-carboxylate methobromide, scopine 9-hydroxy-xanthene-9-carboxylate methobromide, tropenol 9-methyl-xanthene-9-carboxylate-methobromide, scopine 9-methyl-xanthene-9-carboxylate methobromide, tropenol 9-ethyl-xanthene-9-carboxylate methobromide, tropenol 9-difluoromethyl-xanthene-9-carboxylate methobromide and scopine 9-hydroxymethyl-xanthene-9-carboxylate methobromide. Instead of the above-mentioned bromides the compositions according to may also contain for example the corresponding fluorides, chlorides, iodides, sulphates, phosphates, methanesulphonates, nitrates, maleates, acetates, citrates, fumarates, tartrates, oxalates, succinates, benzoates and p-toluenesulphonates, of which the bromides are particularly important.

The above-mentioned compounds may optionally be present in the form of their enantiomers, mixtures of enantiomers or racemates, and optionally in the form of the hydrates and/or solvates thereof.

The concentration in which the above-mentioned anticholinergics are contained in the medicament preparations according to the invention is about 4 to 2000 mg per 100 g, preferably about 8 to 1600 mg per 100 g according to the invention. Particularly preferably, 100g of the formulations according to the invention contain about 80 to about 1360 mg of the above-mentioned anticholinergics (based on pharmacologically active cation). If the above-mentioned bromides are used, the concentration thereof in the compositions according to the invention is usually about 5 to 2500 mg per 100 g, preferably about 10 to 2000 mg per 100 g medicament preparation. Particularly preferably 100 g of the formulations according to the invention contain about 100 to 1700 mg of one of the above-mentioned bromides.

The betamimetics used here are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248, 3-(4-{6-[2-hydroxy-2-(4-hydroxy-3-hydroxymethyl-phenyl)-ethylamino]-hexyloxy}-butyl)-benzyl-sulphonamide, 5-[2-(5,6-diethyl-indan-2-ylamino)-1-hydroxy-ethyl]-8-hydroxy-1H-quinolin-2-one, 4-hydroxy-7-[2-{[2-{[3-(2-phenylethoxy)propyl]-sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino[ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert.-butylamino)ethanol, 6-hydroxy-8-{1-hydroxy-2-[2-(4-methoxy-phenyl)-1,1-dimethyl-ethylamino]-ethyl}-4H-benzo[1,4]oxazin-3-one, 6-hydroxy-8-{1-hydroxy-2-[2-(ethyl 4-phenoxy acetate)-1,1-dimethyl-ethylamino]-ethyl}-4H-benzo [1,4]oxazin-3-one, 6-hydroxy-8-{1-hydroxy-2-[2-(4-phenoxy-acetic acid)-1,1-dimethyl-ethylamino]-ethyl}-4H-benzo[1,4]oxazin-3-one, 8-{2-[1,1-dimethyl-2-(2,4,6-trimethylphenyl)-ethylamino[-1-hydroxy-ethyl}-6-hydroxy-4H-benzo[1,4]oxazin-3-one, 6-hydroxy-8-{1-hydroxy-2-[2-(4-hydroxy-phenyl)-1,1-dimethyl-ethylamino]-ethyl}-4H-benzo[1,4]oxazin-3-one, 6-hydroxy-8-{1-hydroxy-2-[2-(4-isopropyl-phenyl)-1,1dimethyl-ethylamino]-ethyl}-4H-benzo[1,4]oxazin-3-one, 8-{2-[2-(4-ethyl-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid, 8-{2-[2-(3,4-difluoro-phenyl)-1,1-dimethyl-ethylamino]-1-hydroxy-ethyl}-6-hydroxy-4H-benzo[1,4]oxazin-3-one, 1-(4-ethoxy-carbonylamino-3-cyano-5-fluorophenyl)-2-(tert.-butylamino)ethanol, 2-hydroxy-5-(1-hydroxy-2-{2-[4-(2-hydroxy-2-phenyl-ethylamino)-phenyl]-ethylamino}-ethyl)-benzaldehyde, N-[2-hydroxy-5-(1-hydroxy-2-{2-[4-(2-hydroxy-2-phenyl-ethylamino)-phenyl]-ethylamino}-ethyl)-phenyl]-formamide, 8-hydroxy-5-(1-hydroxy-2-{2-[4-(6-methoxy-biphenyl-3-ylamino)-phenyl]-ethylamino}-ethyl)-1H-quinolin-2-one, 8-hydroxy-5-[1-hydroxy-2-(6-phenethylamino-hexylamino)-ethyl]-1H-quinolin-2-one, 5-[2-(2-{4-[4-(2-amino-2-methyl-propoxy)-phenylamino]-phenyl}-ethylamino)-1-hydroxy-ethyl]-8-hydroxy-1H-quinolin-2-one, [3-(4-{6-[2-hydroxy-2-(4-hydroxy-3-hydroxymethyl-phenyl)-ethylamino]-hexyloxy}-butyl)-5-methyl-phenyl]-harnstoff, 4-(2-{6-[2-(2,6-dichloro-benzyloxy)-ethoxy]-hexylamino}-1-hydroxy-ethyl)-2-hydroxymethyl-phenol, 3-(4-{6-[2-hydroxy-2-(4-hydroxy-3-hydroxymethyl-phenyl)-ethylamino]-hexyloxy}-butyl)-benzylsulphonamide, 3-(3-{7-[2-hydroxy-2-(4-hydroxy-3-hydroxymethyl-phenyl)-ethylamino]-heptyloxy}-propyl)-benzylsulphonamide, 4-(2-{6-[4-(3-cyclopentanesulphonyl-phenyl)-butoxy]-hexylamino}-1-hydroxy-ethyl)-2-hydroxymethyl-phenol, N-adamantan-2-yl-2-(3-{2-[2-hydroxy-2-(4-hydroxy-3-hydroxymethyl-phenyl)-ethylamino]-propyl}-phenyl)-acetamide, optionally in the form of their racemates, enantiomers, diastereomers and optionally in the form of their pharmacologically acceptable acid addition salts, solvates or hydrates. The preferred acid addition salts of the betamimetics according to the invention are those selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

The concentration of the above-mentioned betamimetics in the formulations according to the invention is usually about 0.1 to 1600 mg per 100 g, preferably about 0.5 to 1000 mg per 100 g, particularly preferably 0.75 to 200 mg per 100 g. Particularly preferably, 100 g of the formulations according to the invention contain about 1 to about 100 mg of the above-mentioned betamimetics (in each case based on the free base of the above-mentioned compounds).

The corticosteroids used here are preferably compounds selected from among prednisolone, prednisone, butixocortpropionate, flunisolide, beclomethasone, triamcinolone, budesonide, fluticasone, mometasone, ciclesonide, rofleponide, dexamethasone, betamethasone, deflazacort, RPR-106541, NS-126, ST-26, (S)-fluoromethyl 6,9-difluoro-17-[(2-furanylcarbonyl)oxy]-11-hydroxy-16-methyl-3-oxo-androsta-1,4-diene-17-carbothionate, (S)-(2-oxo-tetrahydro-furan-3S-yl) 6,9-difluoro-11-hydroxy-16-methyl-3-oxo-17-propionyloxy-androsta-1,4-diene-17-carbothionate and etiprednol-dichloroacetate, optionally in the form of the racemates, enantiomers or diastereomers thereof and optionally in the form of the salts and derivatives thereof, the solvates and/oder hydrates thereof. Any reference to steroids includes a reference to any salts or derivatives, hydrates or solvates thereof that may exist. Examples of possible salts and derivatives of steroids may be: alkali metal salts, such as for example sodium or potassium salts, sulphobenzoates, phosphates, isonicotinates, acetates, propionates, dihydrogen phosphates, palmitates, pivalates or furoates.

The concentration of the above-mentioned steroids in the formulations according to the invention is usually about 10 to 1800 mg per 100 g, preferably about 100 to 1500 mg per 100 g, particularly preferably 200 to 1000 mg per 100 g. Particularly preferably, 100 g of the formulations according to the invention contain about 400 to about 700 mg of the above-mentioned steroids.

The PDE4-inhibitors used here are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, C1-1018, CDC-801, CDC-3052, D-22888, YM-58997, Z-15370, N-(3.5-dichloro-1-oxo-pyridin-4-yl)-4-difluoromethoxy-3-cyclopropylmethoxybenzamide, (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-R3-cyclopentyloxy)-4-methoxyphenyl]-2-pyrrolidone, 3-(cyclopentyloxy-4-methoxyphenyl)-1-(4-N′-[N-2-cyano-S-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexane-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, 9-cyclopentyl-5,6-dihydro-7-ethyl-3-(2-thienyl)-9H-pyrazolo[3,4-c]-1,2,4-triazolo[4.3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, enantiomers, diastereomers thereof and optionally in the form of the pharmacologically acceptable acid addition salts, solvates or hydrates thereof. Preferred according to the invention are the acid addition salts of the PDE4-inhibitors selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The concentration of the above-mentioned PDE4-inhibitors in the formulations according to the invention is usually about 1 to 1500 mg per 100 g, preferably about 10 to 1200 mg per 100 g, particularly preferably 100 to 1000 mg per 100 g. Particularly preferably, 100 g of the formulations according to the invention contain about 150 to about 800 mg of the above-mentioned PDE4-inhibitors (in each case based on the free base of the above-mentioned compounds).

The LTD4-antagonists used here are preferably compounds selected from among montelukast, pranlukast, zafirlukast, MCC-847 (ZD-3523), MN-001, MEN-91507 (LM-1507), VUF-5078, VUF-K-8707, L-733321, 1-(((R)-(3-(2-(6,7-difluoro-2-quinolinyl)ethenyl)phenyl)-3-(2-(2-hydroxy-2-propyl)phenyl)thio)methylcyclopropane-acetic acid, 1-(((1(R)-3(3-(2-(2.3-dichlorothieno[3.2-b]pyridin-5-yl)-(E)-ethenyl)phenyl)-3-(2-(1-hydroxy-1-methylethyl)phenyl)propyl)thio)methyl)cyclopropane-acetic acid and [2-[[2-(4-tert-butyl-2-thiazolyl)-5-benzofuranyl]oxymethyl]phenyl]acetic acid optionally in the form of the racemates, enantiomers, diastereomers thereof and optionally in the form of the pharmacologically acceptable acid addition salts, solvates or hydrates thereof. Preferred according to the invention are the acid addition salts of the LTD4-antagonists selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists might possibly be capable of forming are meant, for example: alkali metal salts, such as for example sodium or potassium salts, alkaline earth metal salts, sulphobenzoates, phosphates, isonicotinates, acetates, propionates, dihydrogenphosphates, palmitates, pivalates or furoates.

The concentration of the above-mentioned LTD4-antagonists in the formulations according to the invention is usually about 0.1 to 1600 mg per 100 g, preferably about 0.5 to 1000 mg per 100 g, particularly preferably 0.75 to 200 mg per 100 g.

The dopamine agonists used here are preferably compounds selected from among bromocriptine, cabergolin, alpha-dihydroergocryptin, lisuride, pergolide, pramipexol, roxindol, ropinirol, talipexol, terguride and viozan, optionally in the form of the racemates, enantiomers, diastereomers thereof and optionally in the form of the pharmacologically acceptable acid addition salts, solvates or hydrates thereof. Preferred according to the invention are the acid addition salts of the dopamine agonists selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The concentration of the above-mentioned dopamine agonists in the formulations according to the invention is usually about 0.1 to 1500 mg per 100 g, preferably about 1 to 1000 mg per 100 g, particularly preferably 5 to 750 mg per 100 g (in each case based on the free base of the above-mentioned compounds).

The H1-antihistamines used here are preferably compounds selected from among epinastine, cetirizine, azelastine, fexofenadine, levocabastine, loratadine, mizolastine, ketotifen, emedastine, dimetinden, clemastine, bamipine, cexchlorpheniramine, pheniramine, doxylamine, chlorphenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. Preferred according to the invention are the acid addition salts of the H1-antihistamines selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The concentration of the above-mentioned H1-antihistamines in the formulations according to the invention is usually about 1 to 1500 mg per 100 g, preferably about 10 to 1000 mg per 100 g, particularly preferably 20 to 800 mg per 100 g (in each case based on the free base of the above-mentioned compounds).

In another aspect the present invention relates to the use of the medicament formulations according to the invention for preparing a medicament for treating respiratory complaints, selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and chronic obstructive pulmonary disease (COPD), while it is particularly preferable according to the invention to use them for preparing a medicament for the treatment of bronchial asthma and COPD.

It is also preferable to use the medicament combinations according to the invention for preparing a pharmaceutical composition for the treatment of pulmonary emphysema which has its origins in COPD (chronic obstructive pulmonary disease) or α1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the medicament formulations according to the invention for preparing a pharmaceutical composition for the treatment of asthma or COPD. Also of particular importance is the above-mentioned use for preparing a pharmaceutical composition for once-a-day treatment of inflammatory and obstructive respiratory complaints, particularly for the once-a-day treatment of asthma or COPD.

In addition, the present invention relates to a method of treating the above-mentioned diseases, characterised in that one or more of the medicament formulations according to the invention mentioned above are administered in therapeutically effective amounts.

The present invention relates to liquid active substance formulations of these compounds which can be administered by inhalation; the liquid formulations according to the invention have to meet high quality standards. The formulations according to the invention may be inhaled by oral or nasal route. To achieve an optimum distribution of active substances in the lung it makes sense to use a liquid formulation without propellant gases administered using suitable inhalers. Such a formulation may be inhaled by oral or nasal route. Those inhalers which are capable of nebulising a small amount of a liquid formulation in the dosage needed for therapeutic purposes within a few seconds into an aerosol suitable for therapeutic inhalation are particularly suitable. Within the scope of the invention, preferred nebulisers are those in which an amount of less than 100 microlitres, preferably less than 50 microlitres, most preferably less than 25 microlitres of active substance solution can be nebulised preferably in one or two puffs to form an aerosol having an average particle size (or particle diameter) of less than 20 microns, preferably less than 10 microns, so that the inhalable part of the aerosol already corresponds to the therapeutically effective quantity.

An apparatus of this kind for the propellant-free administration of a metered amount of a liquid pharmaceutical composition for inhalation is described in detail for example in International Patent Application WO 91/14468 “Atomizing Device and Methods” and also in WO 97/12687, cf. FIGS. 6a and 6b and the accompanying description. In a nebuliser of this kind a pharmaceutical solution is converted by means of a high pressure of up to 500 bar into an aerosol destined for the lungs, which is sprayed. Within the scope of the present specification reference is expressly made to the entire contents of the literature mentioned above.

In inhalers of this kind the formulations of solutions are stored in a reservoir. It is essential that the active substance formulations used are sufficiently stable when stored and at the same time are such that they can be administered directly, if possible without any further handling, in accordance with their medical purpose. Moreover, they must not contain any ingredients which might interact with the inhaler in such a way as to damage the inhaler or the pharmaceutical quality of the solution or of the aerosol produced.

To nebulise the solution a special nozzle is used as described for example in WO 94/07607 or WO 99/16530 or WO 99/16530. Reference is expressly made here to both these publications.

It is an aim of the present invention to provide an aqueous, ethanolic or aqueous-ethanolic formulation of the compound of formula 1 which meets the high standards needed in order to be able to achieve optimum nebulisation of a solution using the inhalers mentioned hereinbefore. The active substance formulations according to the invention must be of sufficiently high pharmaceutical quality, i.e. they should be pharmaceutically stable over a storage time of some years, preferably at least one year, more preferably two years.

In addition, these propellant-free formulations of solutions must be capable of being nebulised under pressure using an inhaler, the composition delivered by the aerosol produced falling reproducibly within a specified range.

The medicament formulations according to the invention are preferably used in an inhaler of the kind described hereinbefore in order to produce the propellant-free aerosols according to the invention. At this point we should once again expressly mention the patent documents described hereinbefore, to which reference is hereby made.

As described at the beginning, a further developed embodiment of the preferred inhaler is disclosed in WO 97/12687 (cf. in particular FIGS. 6a and 6b and the associated passages of description). This nebuliser (Respimat®) can advantageously be used to produce the inhalable aerosols according to the invention. Because of its cylindrical shape and handy size of less than 9 to 15 cm long and 2 to 4 cm wide, the device can be carried anywhere by the patient. The nebuliser sprays a defined volume of the pharmaceutical formulation out through small nozzles at high pressures, so as to produce inhalable aerosols.

The preferred atomiser essentially consists of an upper housing part, a pump housing, a nozzle, a locking clamp, a spring housing, a spring and a storage container, characterised by
a pump housing fixed in the upper housing part and carrying at one end a nozzle body with the nozzle or nozzle arrangement,
a hollow piston with valve body,
a power take-off flange in which the hollow body is fixed and which is located in the upper housing part,
a locking clamping mechanism located in the upper housing part,
a spring housing with the spring located therein, which is rotatably mounted on the upper housing part by means of a rotary bearing,
a lower housing part which is fitted onto the spring housing in the axial direction.

The hollow piston with valve body corresponds to a device disclosed in WO 97/12687. It projects partially into the cylinder of the pump housing and is disposed to be axially movable in the cylinder. Reference is made particularly to FIGS. 1-4—especially FIG. 31 —and the associated passages of description in the above-mentioned International Patent Application. At the moment of release of the spring the hollow piston with valve body exerts, at its high pressure end, a pressure of 5 to 60 Mpa (about 50 to 600 bar), preferably 10 to 60 Mpa (about 100 to 600 bar) on the fluid, the measured amount of active substance to solution. Volumes of 10 to 50 microlitres are preferred, volumes of 10 to 20 microlitres are more preferable, whilst a volume of 15 microlitres per actuation is particularly preferred.

The valve body is preferably mounted at the end of the hollow piston which faces the nozzle body.

The nozzle in the nozzle body is preferably microstructured, i.e. manufactured by micro-engineering. Microstructured nozzle bodies are disclosed for example in WO 99/16530; reference is hereby made to the contents thereof, especially FIG. 1 disclosed therein and the associated description.

The nozzle body consists for example of two sheets of glass and/or silicon securely fixed together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns and the length being 7 to 9 microns.

If there is a plurality of nozzle openings, preferably two, the directions of spraying of the nozzles in the nozzle body may run parallel to each other or may be inclined relative to one another in the direction of the nozzle opening. In the case of a nozzle body having at least two nozzle openings at the outlet end, the directions of spraying may be inclined relative to one another at an angle of 20 degrees to 160 degrees, preferably at an angle of 60 to 150 degrees, most preferably 80 to 100°.

The nozzle openings are preferably arranged at a spacing of 10 to 200 microns, more preferably at a spacing of 10 to 100 microns, still more preferably 30 to 70 microns. A spacing of 50 microns is most preferred.

The directions of spraying therefore meet in the region of the nozzle openings.

As already mentioned, the liquid pharmaceutical preparation hits the nozzle body at an entry pressure of up to 600 bar, preferably 200 to 300 bar and is atomised through the nozzle openings into an inhalable aerosol. The preferred particle sizes of the aerosol are up to 20 microns, preferably 3 to 10 microns.

The locking clamping mechanism contains a spring, preferably a cylindrical helical compression spring as a store for the mechanical energy. The spring acts on the power take-off flange as a spring member the movement of which is determined by the position of a locking member. The travel of the power take-off flange is precisely limited by an upper stop and a lower stop. The spring is preferably tensioned via a stepping-up gear, e.g. A helical sliding gear, by an external torque which is generated when the upper housing part is turned relative to the spring housing in the lower housing part. In this case, the upper housing part and the power take-off flange contain a single- or multi-speed spline gear.

The locking member with the engaging locking surfaces is arranged in an annular configuration around the power take-off flange. It consists for example of a ring of plastics or metal which is inherently radially elastically deformable. The ring is arranged in a plane perpendicular to the axis of the atomiser. After the locking of the spring, the locking surfaces of the locking member slide into the path of the power take-off flange and prevent the spring from being released. The locking member is actuated by means of a button. The actuating button is connected or coupled to the locking member. In order to actuate the locking clamping mechanism the actuating button is moved parallel to the annular plane, preferably into the atomiser, and the deformable ring is thereby deformed in the annular plane. Details of the construction of the locking clamping mechanism are described in WO 97/20590.

The lower housing part is pushed axially over the spring housing and covers the bearing, the drive for the spindle and the storage container for the fluid.

When the atomiser is operated, the upper part of the housing is rotated relative to the lower part, the lower part taking the spring housing with it. The spring meanwhile is compressed and biased by means of the helical sliding gear, and the clamping mechanism engages automatically. The angle of rotation is preferably a whole-number fraction of 360 degrees, e.g. 180 degrees. At the same time as the spring is tensioned, the power take-off component in the upper housing part is moved along by a given amount, the hollow piston is pulled back inside the cylinder in the pump housing, as a result of which some of the fluid from the storage container is sucked into the high pressure chamber in front of the nozzle.

If desired, a plurality of replaceable storage containers containing the fluid to be atomised can be inserted in the atomiser one after another and then used. The storage container contains the aqueous aerosol preparation according to the invention.

The atomising process is initiated by gently pressing the actuating button. The clamping mechanism then opens the way for the power take-off component. The biased spring pushes the piston into the cylinder in the pump housing. The fluid emerges from the nozzle of the atomiser in the form of a spray.

Further details of the construction are disclosed in PCT applications WO 97/12683 and WO 97/20590, to which reference is hereby made.

The components of the atomiser (nebuliser) are made of a material suitable for their function. The housing of the atomiser and—if the function allows—other parts as well are preferably made of plastics, e.g. by injection moulding. For medical applications, physiologically acceptable materials are used.

FIGS. 6a/b of WO 97/12687 show the Respimat® nebuliser with which the aqueous aerosol preparations according to the invention can advantageously be inhaled. FIG. 6a shows a longitudinal section through the atomiser with the spring under tension, FIG. 6b shows a longitudinal section through the atomiser with the spring released.

The upper housing part (51) contains the pump housing (52), on the end of which is mounted the holder (53) for the atomiser nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58). The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66).

The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomised. The storage container is closed off by the stopper (73), through which the hollow piston projects into the storage container and dips its end into the fluid (supply of active substance solution).

The spindle (74) for the mechanical counter is mounted on the outside of the spring housing. The drive pinion (75) is located at the end of the spindle facing the upper housing part. On the spindle is the slider (76).

The nebuliser described above is suitable for nebulising the aerosol preparations according to the invention to form an aerosol suitable for inhalation.

If the formulation according to the invention is nebulised using the technology described above (Respimat®), the mass expelled, in at least 97%, preferably at least 98% of all the actuations of the inhaler (puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

The formulation according to the invention can also be nebulised using inhalers other than those described above, for example jet-stream inhalers. The following are examples of devices in which the formulations according to the invention may be used. They are, for example, devices according to International Patent Applications WO 02/51466, WO 03/49792 and WO 04/22242 (Chrysalis), devices according to International Patent Applications WO 94/14543, WO 00/35524, WO 00/38770 and WO 00/64590 (Battelle/Ventaira), devices according to the publications US 20060048772, US 20050224076 and WO 05/42075 (Pari), devices according to the publications WO 94/16755, WO 94/16717, WO 96/13291, WO 96/13161, WO 98/22169, WO 98/33480, WO 98/48878 and WO 02/74375 (Aradigm) as well as devices according to the publication EP 1211628 (Canon).

The present invention also relates to an inhalation kit consisting of one of the pharmaceutical preparations according to the invention described above and an inhaler suitable for nebulising this pharmaceutical preparation. The present invention preferably relates to an inhalation kit consisting of one of the pharmaceutical preparations according to the invention described above and the Respimat® inhaler described above.

The examples of formulations given below serve as illustrations without restricting the subject matter of the present invention to the compositions shown by way of example.

In the examples of formulations that follow, BAC denotes benzalkonium chloride and EDTA denotes disodium edetate-dihydrate. The percentages specified are percent by weight while the ethanol content is given in percent by volume.

A) Tiotropium Bromide Formulations

100 g medicament formulation (pH adjusted in each case with HCl to 2.9±0.2) contain in purified water or water for injections:

| | ethanol | BAC | EDTA | | tiotropium* |
|---|---|---|---|---|---|
| Example | (Vol. %) | (mg) | (mg) | excipient (%) | (%) |
| 1 | 0 | 10 | 50 | NaCl (10) | 0.1 |
| 2 | 0 | 10 | 50 | NaCl (15) | 0.2 |
| 3 | 0 | 5 | 30 | lactose (5) | 0.2 |
| 4 | 0 | 5 | 30 | KCl (10) | 0.2 |
| 5 | 0 | 5 | 20 | NaCl (10) | 0.2 |
| 6 | 0 | 5 | 40 | KCl (10) | 0.1 |
| 7 | 20 | 10 | 30 | mannitol (10) | 0.2 |
| 8 | 40 | 5 | 50 | NaCl (5) | 0.2 |
| 9 | 0 | 10 | 10 | NaCl (10) | 0.023 |
| 10 | 0 | 10 | 10 | NaCl (15) | 0.045 |

*based on free cation

B) Ipratropium Bromide Formulations

100 g medicament formulation (pH adjusted in each case with HCl to 3.2±0.4) contain in purified water or water for injections:

| | ethanol | BAC | EDTA | | ipratropium* |
|---|---|---|---|---|---|
| Example | (Vol. %) | (mg) | (mg) | excipient (%) | (%) |
| 1 | 0 | 5 | 40 | NaCitrate (10) | 1.0 |
| 2 | 0 | 5 | 40 | Lactose (5) | 1.0 |
| 3 | 80 | 5 | 20 | sorbitolol (10) | 1.0 |
| 4 | 95 | 5 | 20 | sorbitolol (4) | 1.0 |
| 5 | 0 | 10 | 50 | Na₃PO₄ (10) | 1.0 |
| 6 | 0 | 10 | 50 | NaHCO₃ (15) | 1.0 |
| 7 | 0 | 10 | 50 | KCl (10) | 1.0 |

*based on ipratropium bromide monohydrate

C) Fenoterol formulations

100 g medicament formulation (pH adjusted in each case with HCl to 3.2±0.4) contain in purified water or water for injections:

| | ethanol | BAC | EDTA | | Fenoterol* |
|---|---|---|---|---|---|
| Example | (Vol. %) | (mg) | (mg) | excipient (%) | (%) |
| 1 | 60 | 5 | 30 | sorbitolol (15) | 1.0 |
| 2 | 0 | 5 | 10 | Glucose (10) | 1.0 |
| 3 | 0 | 10 | 50 | Glucose (10) | 1.0 |

*based on hydrobromide

Determining the Droplet Sizes

The particle size distribution or mean volumic diameter is determined by laser diffraction, sing a particle size measuring apparatus made by Sympatec, model Helos BF, with a volume flow of 28.3 l/min and under climatic conditions of approx. 23° C./approx. 100% relative humidity.

In order to produce the approx. 100% relative humidity a compressed air source is connected to a moistening apparatus filled with water. The compressed air flows through the water-filled moistening apparatus and thus accumulates moisture. The outlet from the moistening apparatus is connected through a hose to a moisture sensor which analyses the current humidity of the air in the air flow. The moisture sensor is connected by a hose to the adapter for the Respimat® inhaler. This is connected to the modified Sample Induction Port. The modified Sample Induction Port is installed in the measuring zone of the particle size measuring apparatus and constitutes the actual measuring chamber. To produce the air current, the outlet of the modified Sample Induction Port is connected via a suitable adapter to a suction apparatus, while a catching apparatus is interposed between them to catch the dose released during measurement. The suction apparatus is connected to a vacuum source.

To determine the particle size distribution of mean volumic diameter the particle size measuring apparatus is operated with an R3 lens, focal length 100 mm. An optical concentration of >0.1% to channel 30 is set as the trigger condition for the measurement. Evaluation is carried out using the MIE theory with the associated substance parameters.

Carrying Out the Measurement:

The Respimat® inhaler is tensioned, the mouthpiece, lock nut and nozzle are wiped dry and then placed in the adapter. The moisture supply is connected up, and at a constant humidity of about 100% r.h. A reference measurement is carried out. By actuating the trigger button on the Respimat® inhaler the aerosol is produced. The aerosol delivered is detected by the particle size measuring apparatus and its particle size distribution or the mean volumic diameter is calculated.

The following Table shows the droplet sizes measured for the formulation examples given above.

| Example | D(v; 50)* |
|---|---|
| A.1 | 2.0 ± 0.2 |
| A.2 | 2.3 ± 0.4 |
| A.3 | 1.6 ± 0.2 |
| A.4 | 2.0 ± 0.4 |
| A.5 | 2.0 ± 0.4 |
| A.6 | 2.0 ± 0.4 |
| A.7 | 2.0 ± 0.4 |
| A.8 | 1.6 ± 0.2 |
| B.1 | 2.1 ± 0.4 |
| A.9 | 2.0 ± 0.3 |
| A.10 | 2.3 ± 0.2 |
| B.2 | 1.7 ± 0.2 |
| B.3 | 2.1 ± 0.2 |
| B.4 | 1.6 ± 0.2 |
| B.5 | 2.1 ± 0.2 |
| B.6 | 2.3 ± 0.2 |
| B.7 | 2.1 ± 0.1 |
| C.1 | 2.3 ± 0.3 |
| C.2 | 2.1 ± 0.4 |
| C.3 | 2.1 ± 0.4 |

*D(v; 50) denotes: mean volumic diameter;

By comparison, the following values were determined for formulations without any additional inert excipient:

| | ethanol | BAC | EDTA | active substance | |
|---|---|---|---|---|---|
| Example | (%) | (mg) | (mg) | (%) | D(v; 50) |
| Rev. 1 | 0 | 10 | 50 | tiotropium (0.1) | 0.5 ± 0.1 |
| Rev. 2 | 0 | 10 | 10 | tiotropium | 0.4 ± 0.2 |
| | | | | (0.045) | |
| Rev. 3 | 95 | 10 | 10 | fenoterol (1.0) | 0.9 ± 0.4 |

What is claimed is:

1. Solution formulation for inhalation which contain one or more active substances in a solvent selected from among water, ethanol and water-ethanol mixtures; and
at least one inert, non-volatile excipient in an amount such that the total concentration of non-volatile constituents in the formulation is ≧3 wt. %.

2. Solution formulation according to claim 1, characterised in that the one or more active substances is selected from anticholinergics, betamimetics, corticosteroids, PDE4-inhibitors, LTD4-antagonists, dopamine agonists and H1-antihistamines.

3. Solution formulation according to claim 1, characterised in the at least one inert, non-volatile excipients is a preservatives or a complexing agent.

4. Solution formulation according to claim 1, characterised in that the at least one inert, non-volatile excipient is selected from monosaccharides, disaccharides, oligo- and polysaccharides, polyalcohols, or a salt thereof.

5. Solution formulation according to claim 1, characterised in that the total concentration of non-volatile constituents in the formulation is ≧5 wt. %.

6. Solution formulation according to claim 1, characterised in that the total concentration of non-volatile constituents in the formulation is ≧10 wt. %.

7. Solution formulation according to claim 1, characterised in that the solvent contains pure water.

8. Solution formulation according to claim 1, characterised in that the solvent contains ethanol or mixtures of ethanol and water.

9. Solution formulation according to claim 8, characterised in that the solvent contains a mixture of ethanol and water, wherein the percentage proportion of ethanol by weight is in the range between 5 and 99%.

10. Solution formulation according to claim 8, characterised in that the solvent contains a mixture of ethanol and water, wherein the percentage proportion of ethanol by weight is in the range between 10 to 96%.

11. Solution formulation according to claim 1, characterised in that the pH of the formulation is in the range from 2.0 to 6.5.

12. Use of a solution formulation according to claim 1 for the treatment of a respiratory complaint selected from obstructive pulmonary diseases of various origins, pulmonary emphysema of various origins, restrictive pulmonary diseases, interstitial pulmonary diseases, cystic fibrosis, bronchitis of various origins, bronchiectasis, adult respiratory distress syndrome, and all forms of pulmonary oedema.
